(19) United States
(12) Patent Application Publication
(10) Pub. No.: US 2005/0096681 A1
(43) Pub. Date: May 5, 2005

(54) Medical device for electrotomy

(75) Inventors: Kai Desinger, Berlin (DE); Andre Roggan, Berlin (DE); Thomas Stein, Berlin (DE)

Correspondence Address: BECK AND TYSVER, 2900 THOMAS AVENUE SOUTH, SUITE 100, MINNEAPOLIS, MN, 55416, US

(73) Assignee: Celon AG Medical Instruments

(21) Appl. No.: 10/973,893

(22) Filed: Oct. 26, 2004

(30) Foreign Application Priority Data
Oct. 29, 2003 (DE) 103 51 818.5
Dec. 16, 2003 (DE) 103 61 142.8

(51) Int. Cl. A61B 17/32

(52) U.S. Cl. 606/169

(57) ABSTRACT

A medical device assembly for cutting body tissue by means of electrotomy, includes a generator for a high frequency alternating current which can be delivered by way of a pole and an opposite pole, wherein each pole is connected to at least one respective output terminal of the generator; a gripping instrument with two gripping arms which each have at least one respective gripping surface and which are connected together in such a way that the gripping surfaces are movable towards each other, wherein the gripping surface is at least partially electrically conductive and each is individually electrically connected to a respective terminal or they are electrically connected to a common terminal for an electrical supply line; and a cutting instrument which has a handle and a cutting electrode, wherein in operation the cutting electrode is connected to a pole of the generator and both gripping surfaces are connected to the opposite pole of the generator.

This application claims priority to German patent application, Ser. No. 103 51 818.5, filed Oct. 29, 2003 and German patent application, Ser. No. 103 61 142.8, filed Dec. 16, 2003.

### FIELD OF INVENTION

The invention concerns a medical device assembly and a method of cutting, separating or removing body tissue by means of electrotomy. The medical device assembly includes a generator for a high frequency alternating current and a cutting instrument. The invention also concerns a gripping instrument for use with the medical device assembly, a cutting instrument for the medical device assembly and a corresponding converter unit.

### BACKGROUND OF THE INVENTION

The use of electrosurgical methods for tissue separation or tissue removal (electrotomy) has already been a routine procedure in surgery for decades. Such methods afford the advantage that the tissue is severed in the form of a so-called fusion cut in which the cutting electrode produces a spark discharge which causes vaporization of the tissue in the immediate proximity of the cutting electrode and immediately closes off small capillary vessels so that it is possible to cut tissue in an almost blood-free fashion.

That method is used in all surgical and other medical disciplines for tissue separation or tissue removal. The electrosurgical application technology is used in accordance with the state of the art in the so-called monopolar use technology; that is to say, besides the cutting electrode which is in the form of an active electrode, a large-area return electrode (also referred to as the neutral electrode) is fitted to the extremities of the patient being treated in order to guarantee a high frequency current flow. That monopolar use procedure suffers from the disadvantage that the current flows by way of the entire patient. The disadvantage arises on the one hand out of the fact that the monopolar use procedure represents an inherent risk potential for the patient and the user as the current flows through the entire patient and burns due to roving leakage currents and a neutral electrode cannot be excluded. The above-indicated disadvantage further arises out of the fact that a part of the energy required for electrotomy, in particular spark discharge, is converted into heat on its way to the neutral electrode so that the efficiency of a corresponding arrangement is poor.

In recent years therefore investigations have been conducted into some concepts for electrosurgical tissue separation using a bipolar application technology, but these cannot compete with the quality of a monopolar system. That is due to the fact that, with the bipolar procedure, both electrodes must be arranged one beside the other in the direct proximity in the target region of the intervention. As however for physical reasons there is only ever one electrode, even if of the same geometry, which can become an active electrode at which the sparks are produced, the other electrode is virtually forced to act as the return electrode, and that results in a greatly reduced quality of incision.

### SUMMARY OF THE INVENTION

Therefore the object of the present invention is to provide a medical device assembly and components for cutting body tissue, which as far as possible avoids the above-mentioned disadvantages. The invention equally seeks to provide components for an arrangement of that nature.

In accordance with the invention that object is attained by a medical device assembly for cutting body tissue by means of electrotomy, which comprises
a generator for a high frequency alternating current which can be delivered by way of a pole and an opposite pole, wherein each pole is connected to at least one respective output terminal of the generator,
a gripping instrument with two gripping arms which each have at least one respective gripping surface and which are connected together in such a way that the gripping surfaces are movable towards each other, wherein the gripping surface is at least partially electrically conductive and each is individually electrically connected to a respective terminal or both gripping surfaces are electrically connected to a common terminal for an electrical supply line, and
a cutting instrument which has a handle and a cutting electrode, wherein in operation the cutting electrode is connected to a pole of the generator and both gripping surfaces are connected to the opposite pole of the generator.

The gripping instrument, for example is in the form of a pair of tweezers which, with the corresponding gripping surfaces thereof, represents a neutral electrode, while the cutting instrument itself has a cutting electrode which in this configuration is in the form of an active electrode. As usually for example in the case of cutting back an uvula the severed tissue must at the same time in any case be gripped, the gripping instrument in the arrangement according to the invention elegantly performs a dual function.

Preferably, the above-described medical device assembly has a converter unit which is connected between the generator and the gripping instrument or between the generator and the cutting instrument respectively and which is connected at the generator side to the pole and the opposite pole of the generator and at the output side to the gripping instrument on the one hand and the cutting instrument on the other hand, in such a way that at a given moment in time both gripping surfaces of the gripping instrument involve a common first potential and the cutting electrode involves a second potential.

Such a converter unit makes it possible to use generators which are already available on the market for the electrotomy assembly which is set forth here and in which output and input impedances of the generator on the one hand and the rest of the assembly on the other hand can be matched to each other by the converter unit.

Independent inventive aspects are embodied in a gripping instrument for a medical device assembly of the above-indicated kind, having two gripping arms which each have at least one respective gripping surface and which are connected together in such a way that the gripping surfaces are movable towards each other and the gripping instrument can selectively assume an open or a closed condition. In that case each gripping surface is of an at least electrically conductive native and is electrically insulated in relation to respective other gripping surface. In a first variant of the gripping instrument both gripping surfaces are each individually electrically connected to a respective terminal for a respective electrical supply line so that in the first variant the gripping instrument is to be operated in the form of a bipolar instrument if different potentials are applied to the two gripping surfaces. For operation in conjunction with the cutting instrument however the same potential is preferably applied to both gripping surfaces so that in the first variant in this case the gripping instrument is operated virtually in a monopolar mode.

An important feature of the gripping instrument is that the electrical supply line thereof is connected by way of a line branch on the one hand to an electrical supply line for the cutting instrument and on the other hand to a common electrical supply line for the gripping instrument and the cutting instrument, which leads to a generator or a converter unit.

Alternatively both gripping surfaces of the gripping instrument may also be connected to a common terminal for an electrical supply line so that, in this second variant, the gripping instrument is to be operated only in a unipolar or monopolar mode.

Preferred variants of the two above-indicated variants of the gripping instrument have gripping surfaces with a surface structure having such raised portions that body parts can be securely gripped. The raised portions are for example in the form of biting teeth, that is to say of a pointed shape which can penetrate into the body tissue in a similar manner to a crampon. That is particularly advantageous for the reason that body tissue to be removed is frequently both moist and also soft so that basically it is difficult to grip.

The gripping surfaces are preferably a component part of a gripping attachment which is to be interchangeably secured to the respective gripping instrument and which is preferably to be clamped onto a respective distal end of a gripping arm. That makes it possible, after an operation, for essential parts of the gripping instrument to be used again and for only the gripping attachments themselves to be replaced by new ones, for sterility reasons.

Suitable and preferred configurations of the gripping instrument include the configuration of the gripping instrument in the manner of a pair of tweezers or in the manner of a pair of biting tongs.

It is preferred in that respect if the gripping instrument automatically assumes the opened condition without an external force acting thereon, as is known from tweezers. The gripping instrument is then to be closed for example against a spring force.

It may also be advantageous if the gripping instrument is so designed that, after being set in the closed condition, it retains the closed condition or is to be locked in the closed condition. That makes it possible for the gripping instrument to be secured to a body part, without having to be permanently held fast.

The latter effect can also be achieved if, in contrast to known tweezers, the gripping instrument assumes the closed condition of its own accord and has to be opened for attaching the gripping instrument to a part of the body. In this variant the gripping instrument automatically applies a clamping force to the body tissue.

A further independent aspect of the invention concerns a cutting instrument for a medical device assembly of the kind set forth in the opening part of this specification, in which the cutting electrode is arranged at a distal end of the electrode shaft and is connected to the handle by way of the electrode shaft. In that arrangement the cutting electrode is preferably pointed. Alternatively the cutting electrode can also be in the form of a wire loop. Such a cutting instrument permits clean tissue incisions by the spark discharge emanating from the cutting electrode.

It is a particular feature of the cutting instrument that the electrical supply line thereof is connected by way of a branching on the one hand to the electrical supply line to the gripping instrument and on the other hand to a common electrical supply line for the gripping and cutting instrument, which leads for example to a generator or a converter unit.

The handle of the cutting instrument preferably has a gripping surface which is insulated with respect to the cutting electrode. In addition the handle has a terminal for an electrical supply line, with which the cutting electrode is to be electrically connected to an electric line leading to the generator or the converter unit.

In addition, in the cutting instrument, the cutting electrode or the cutting electrode together with the electrode shaft is preferably replaceably connected to the rest of the cutting instrument, in particular the handle. In that way, after an operation, only the cutting electrode or the electrode shaft together with the cutting electrode has to be replaced while the rest of the cutting instrument can be re-used.

In addition, a press switch which is to be actuated with a finger can be provided on the cutting instrument, the switch being arranged on the handle and being adapted to selectively make or break an electrical connection to the cutting electrode.

Instead of or in addition to a press switch on the cutting instrument it is also possible to provide a foot switch which is connected to a corresponding generator or control device in such a way that the high frequency current is to be switched on and interrupted by means of the foot switch. The foot switch is preferably so designed that, when the foot switch is pressed, a potential is applied to the cutting instrument and the gripping instrument and when the foot switch is released the corresponding current is interrupted.

In regard to the generator for the medical device assembly as set forth in the opening part of this specification, preferred generators are those which have a protective device which responds to impedance between the pole and the opposite pole falling below or rising above a critical limit value. In that way it is possible to implement automatic shut-down of the generator in the case of a short-circuit (when the impedance value falls below a critical value) or after the end of the cutting operation (when a critical impedance value is exceeded).

The converter unit referred to in the opening part of this specification preferably has a transformer which is to be connected at the input side to two poles of the generator and which has a corresponding primary winding and which at the output side has a secondary winding which is connected on the one hand to a first terminal for the cutting instrument and on the other hand to at least one second terminal for the gripping instrument. Two terminals for the gripping instrument on the corresponding side of the secondary winding make it possible for the above-described bipolar first variant of the gripping instrument also to be operated in a monopolar mode.

Preferably capacitors are arranged between the secondary winding and respective terminals for the cutting instrument on the one hand and the gripping instrument on the other hand.

The invention will be described in greater detail below by means of embodiments.

### BRIEF DESCRIPTION OF THE DRAWINGS

An exemplary version of a medical device is shown in the figures, wherein like reference numerals refer to equivalent structure throughout, and wherein:

FIG. 1 shows a medical device assembly according to the invention,

FIG. 2 shows an overview of an apparatus for bipolar cutting of biological tissue,

FIG. 3 shows a circuit diagram of a converter unit for an apparatus as shown in FIG. 1,

FIG. 4 shows a medical device assembly similar to FIG. 1 with an external converter unit,

FIG. 5 shows a distal end of a gripping arm of a gripping instrument with a gripping attachment fitted thereto,

FIGS. 6a through c show alternative distal ends of the gripping arms of a gripping instrument,

FIG. 7 shows an alternative cutting instrument in the form of a pair of coagulating shears,

FIG. 8 is a view by way of example of use of the apparatus of FIG. 2, and

FIG. 9 is a diagrammatical representation of an alternative arrangement of the apparatus.

### DETAILED DESCRIPTION OF THE PREFERRED EMBODIMENT(S)

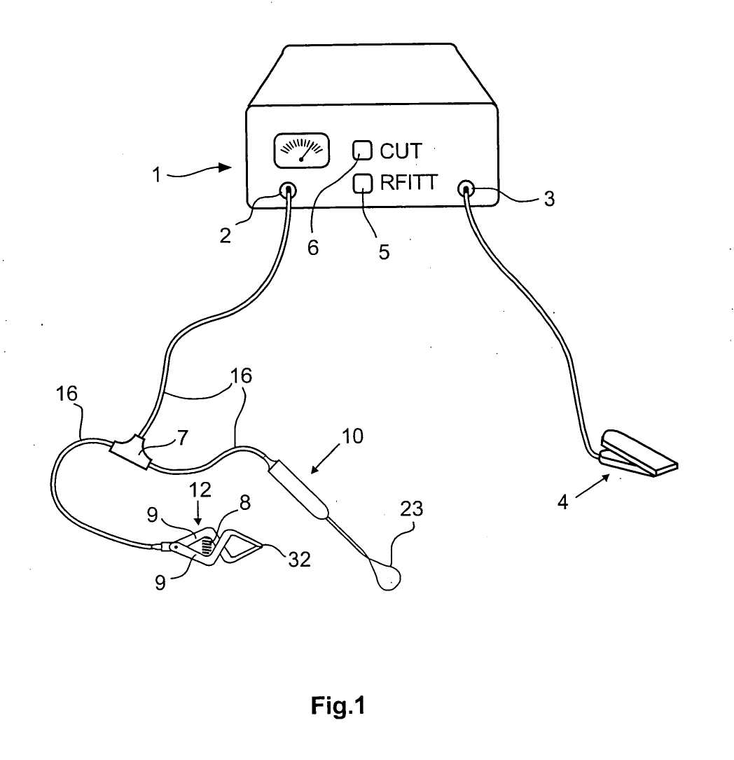

The medical device assembly shown in FIG. 1 has a control device 1 with integrated generator. The control device has a control terminal 3 to which a foot switch 4 is connected. In addition the control device 1 has a bipolar terminal 2, to which on the one hand a cutting instrument 10 and on the other hand a gripping instrument 12 are connected by way of electrical supply lines 16 and a line branching 7, more specifically in such a way that a high frequency alternating current is applied in bipolar mode between a cutting electrode 23 of the cutting instrument 10 on the one hand and gripping surfaces 32 of the gripping instrument 12 as a counterpart electrode on the other hand as soon as the foot switch 4 is actuated.

The control device 1 includes a generator and press switches 5 and 6 which provide that, depending on which of the two switches is actuated, the bipolar output 2 involves either an alternating current which is required for interstitial thermotherapy (RFITT) with a maximum peak amplitude of 80 V or an alternating current with a maximum peak amplitude of 200 V, which is suitable for the cutting operation.

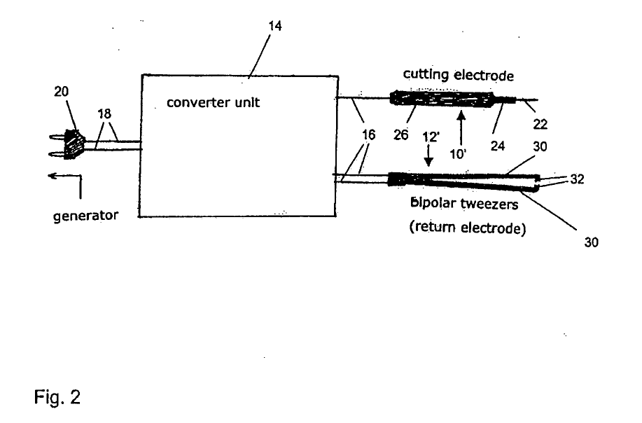

FIG. 2 shows those component parts of a medical device assembly which are to be connected to a generator for a high frequency alternating current. The generator itself is not shown.

FIG. 2 shows on the one hand a cutting instrument 10′ and a gripping instrument 12′ in the form of tweezers. FIG. 2 also diagrammatically shows a converter unit 14 to which on the one hand the cutting instrument 10′ and on the other hand the gripping instrument 12′ are connected by way of corresponding electrical supply lines 16. At the input side, the converter unit 14 has a cable 18 with a connector plug 20 connected thereto for connecting the converter unit 14 to a generator.

The cutting instrument 10′ has a cutting electrode 22 which is arranged distally on an electrode shaft 24 and connected by way of the electrode shaft 24 to a handle 26 of the cutting instrument. The cutting electrode 22 together with the electrode shaft 24 are releasably connected to the handle 26 so that the cutting electrode 22 with electrode shaft 24 is to be replaced after an operation while the handle 26 is to be re-used. Disposed on the handle 26 is a terminal for that electrical supply line 16 with which the cutting instrument 10 is connected to the converter unit 14. That electrical connection is releasable. Also arranged in the region of the handle is a press switch which is to be operated by a finger and which can make or break an electrical connection to the cutting electrode 22.

Alternatively to a bar-shaped pointed cutting electrode 22, it is also possible to provide a cutting electrode in the form of a wire loop 23, as is shown in FIG. 1.

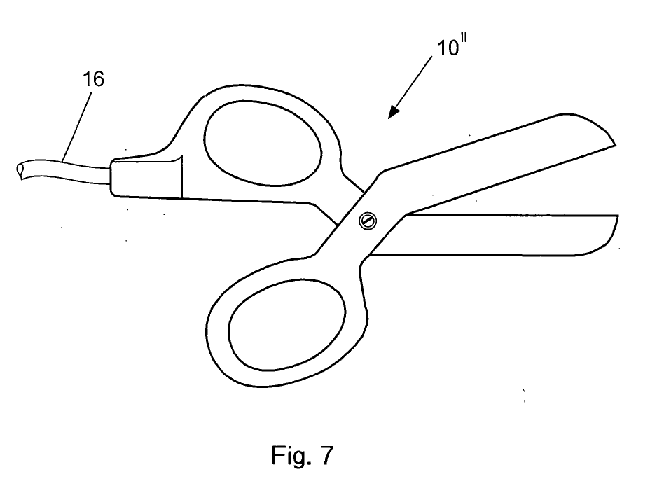

As another alternative, FIG. 7 shows an alternative cutting instrument in the form of a pair of coagulating shears 10″. The shears 10″ are to be connected to the control device 1 by way of an electrical supply line 16, with the consequence that an electrical potential is to be applied to the cutting edges of the shears. That makes it possible to cut with the shears 10″, as by conventional shears, while at the same time, by virtue of the applied potential, in the region of the cut, tissue sclerosis (coagulation) occurs, which immediately closes any blood vessels which have possibly been cut.

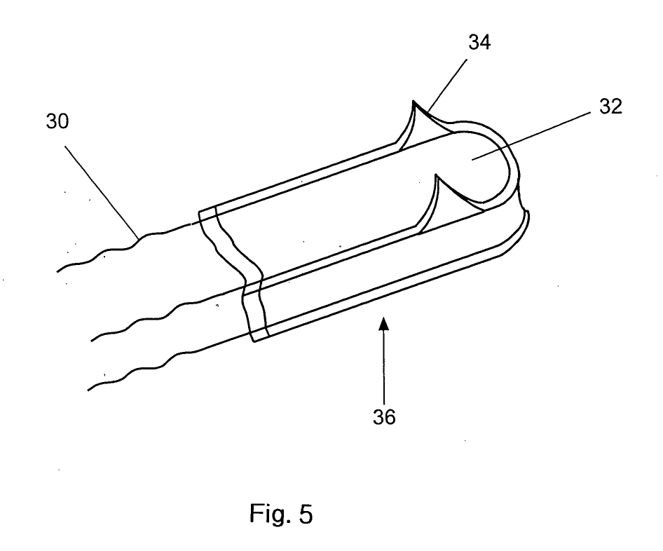

As shown in FIG. 2, the gripping instrument 12′ is in the form of bipolar tweezers, in accordance with the above-described first variant, and is therefore connected by way of two electrical supply lines 16 to a pole of the converter unit 14 and by way of the converter unit 14 to a pole of the generator. The gripping instrument 12′ has two gripping arms 30 which have mutually facing gripping surfaces 32 at the distal ends. Each of the two gripping surfaces 32 is electrically connected to one of the electrical supply lines 16 and is of an electrically conductive nature at least in portions thereof. The gripping surfaces 32 have raised portions (not visible in FIG. 1) in the form of biting teeth 34, as can be seen from FIG. 5. It can further be seen from FIG. 5 that a respective gripping surface 32 together with the biting teeth 34 are a component part of a gripping attachment 36 which comprises high-quality steel and which is to be pushed onto the distal end of a respective gripping arm 32 of the gripping instrument 12. In that way, after an operation, the corresponding gripping attachments can be replaced and the rest of the gripping instrument 12 can be used again.

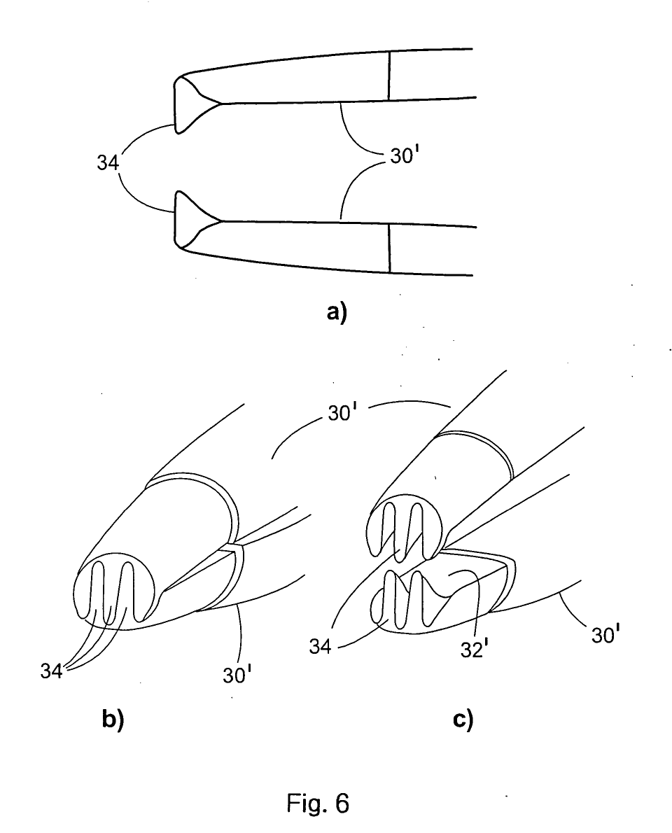

FIG. 6 shows an alternative configuration of the distal end of the gripping arms 30′ of a gripping instrument 12 or 12′, as is illustrated in FIG. 1 or FIG. 2. FIG. 6a shows a side view of the gripping arms 30′. FIG. 6b and 6c show perspective views of the distal end of the gripping arms 30′ on the one hand in the closed condition and on the other hand in the open condition.

In an alternative variant the gripping instrument is not in the form of conventional tweezers but in the form of self-closing tweezers, as diagrammatically shown in FIG. 1 in the form of the gripping instrument 12. A compression spring 8 provides that the gripping instrument 12 closes automatically and can be opened against the force of the compression spring 8 by pressure applied to the gripping surfaces 9. A gripping instrument 12 of that nature affords the advantage that the physician performing the procedure, with the hand guiding the gripping instrument 12, does not simultaneously constantly have to apply the required holding force.

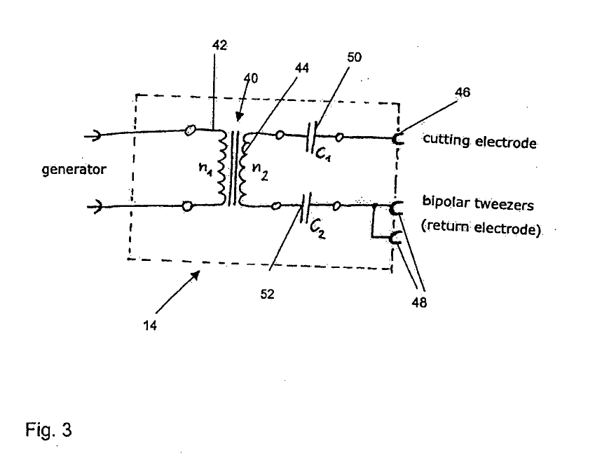

FIG. 3 shows a circuit diagram of the converter unit 14 from which it can be seen that the converter unit 14 has a transformer 40 having a primary winding 42 which is intended for connection to the generator and a secondary winding 44 which is connected on the one hand to a terminal 46 for the cutting instrument and on the other hand to two parallel-connected terminals 48 for the gripping instrument. A first capacitor 50 is arranged between the secondary winding 44 and the cutting electrode 46. A second capacitor 52 is arranged on the other side of the secondary winding between the secondary winding 44 and the terminals 48. The first and second capacitors 50 and 52 can be interchanged for impedance matching purposes. The capacitors further have the effect of suppressing dc components and thus Faraday effects.

The transformer 40 is moreover a high frequency transformer in which the number of turns n₂ of the secondary winding 44 is greater than the number of turns n₁ of the primary winding 42, i.e. n₂>n₁. A high frequency transformer of that kind steps up a low output voltage of a usual generator (about 80 V), which is designed for the purposes of tissue coagulation, into high voltage values which permit igniting of a spark of a cutting electrode and thus severing of biological tissue. The output voltage at the converter unit 14 is for example about 200 V.

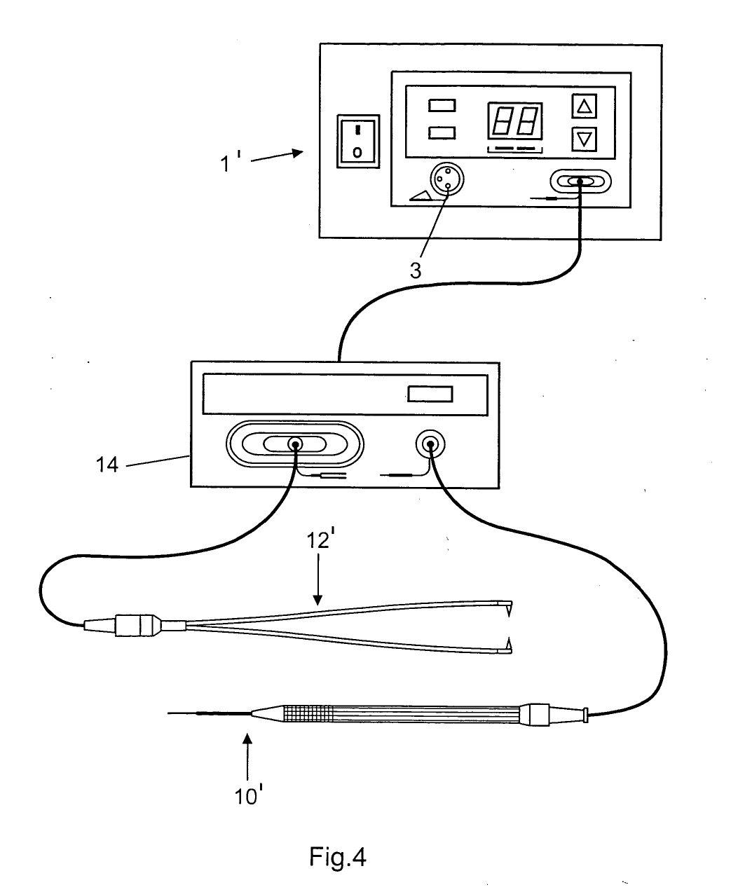

FIG. 4 shows a medical device assembly similar to FIG. 1, in which a converter unit 14, as illustrated in FIGS. 2 and 3, is connected between the control device 1′ on the one hand and the gripping and cutting instrument 12 and 10′ respectively on the other hand. In this arrangement the converter unit 14 performs the function of the line branching (7) shown in FIG. 1. In the embodiment illustrated in FIG. 4 the gripping instrument 12 is in the form of tweezers with biting teeth (see also FIG. 5) and the cutting instrument 10′ is shown with a pointed cutting electrode. Instead of those instruments however it would also be possible to use any of the cutting and gripping instruments illustrated herein.

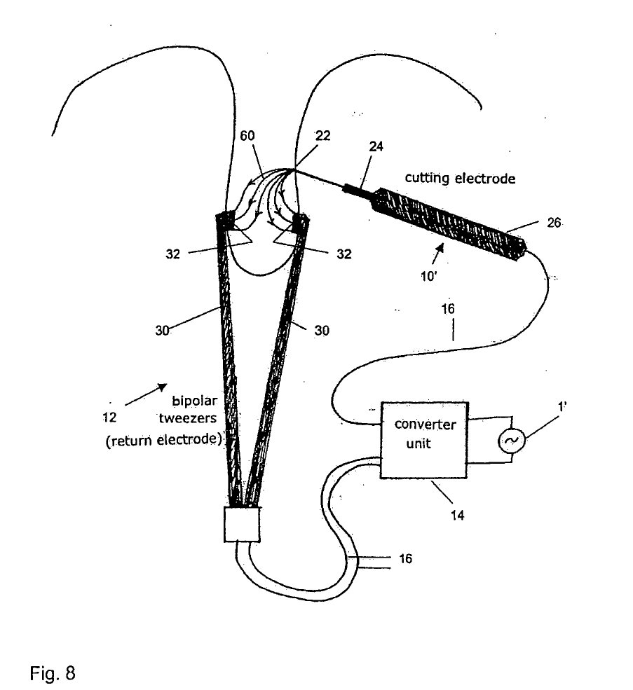

Finally FIG. 8 shows a situation of use for the above-described medical device assembly, more especially for the specific example of cutting back an uvula. With the gripping surfaces 32, the gripping instrument 12 grips the uvula. As shown in FIG. 2 the gripping instrument 12 is connected in a quasi-monopolar mode to the converter unit 14 and with its gripping surfaces 32 forms a neutral electrode.

Connected to the second pole of the converter unit 14 is the cutting instrument 10 so that, upon actuation of the corresponding press switch, an electrical connection is made between the corresponding pole of the converter unit 14 and the cutting electrode 22 of the cutting instrument 10. That then affords the flow of current, diagrammatically illustrated by arrows 60, from the cutting electrode 22 to the gripping surfaces 32, in which case, by virtue of field strength concentration, the cutting electrode 22 is in the form of an active electrode and a spark discharge occurs between the tip of the cutting electrode 22 and the body tissue to be severed.

In this arrangement the converter unit 14 is connected the whole time to the symbolically indicated generator 1′ which has a control unit which causes the current to be switched off in the event of a short circuit or when a maximum impedance limit value is exceeded.

For the situation of use illustrated in FIG. 8, the gripping instrument used, also in accordance with the above-described second variant, can be in the form of a unipolar gripping instrument in which both gripping surfaces 32 are connected to a common electrical terminal.

A method of using the above-described medical device assembly provides that firstly the assembly is put together as shown by way of example in FIG. 8. Next the physician grips the piece of tissue to be removed, using the gripping instrument 12. As the gripping instrument 12 is attached as an opposite electrode to the piece of tissue to be removed, a flow of current occurs essentially only in the piece of tissue to be removed so that potential burns due to poor electrical contact for example between the gripping instrument 12 and the piece of tissue to be removed do not cause problems.

Then the physician guides the cutting instrument 10 and more precisely the cutting electrode 22 into the region of the intended tissue incision. By pressing on the corresponding press switch he can make the electrical connection between the cutting electrode 22 and the corresponding pole of the converter unit 14 so that, when the cutting electrode 22 sufficiently approaches the body tissue, a spark gap is formed between the cutting electrode 22 and body tissue, by which the body tissue is severed. The corresponding current flows between the cutting electrode through the piece of body tissue to be severed, to the gripping surfaces 32 of the gripping instrument, which act as a neutral electrode by virtue of the larger surface area thereof. During that operation the physician firmly holds the piece of body tissue to be removed, with the gripping instrument 12, so that he can remove it after complete separation from the rest of the body tissue, by means of the gripping instrument 12. The electrotomy procedure is concluded at that stage.

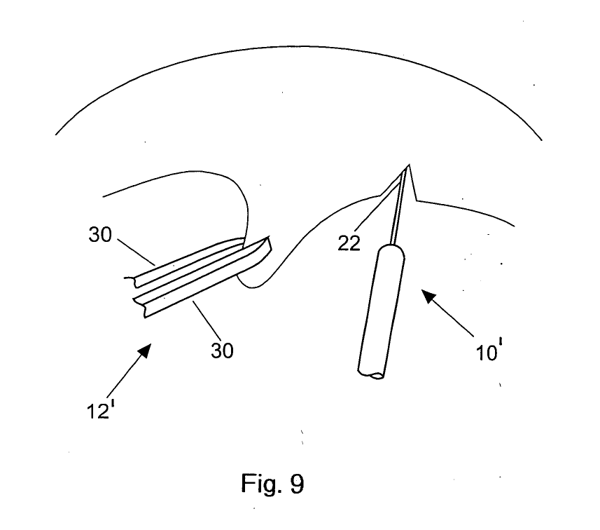

FIG. 9 shows how the above-described medical device assembly can be used for example for incisions in a velum. For that purpose the uvula is pulled in a direction (towards the left in FIG. 9) with the gripping instrument 12′ and at the same time an incision is made with the cutting instrument 10′. The pull applied with the gripping instrument 12′ causes the incision to open up, as shown in FIG. 9.

As an alternative to the cutting back of an uvula (uvulapalatopharyngoplasty; UPPP), illustrated by way of example in FIG. 8, it is also possible for example to cut back a velum for the treatment of rhonchopathy. Further areas of use can also be envisaged. Such further areas of use include removal or reduction of tonsils. In addition the medical device assembly can also be used in dermatology, for example for the treatment of warts. A wart can be gripped for example with a gripping instrument and can then be cut away from the rest of the tissue by a peeling action with a cutting instrument for example with a wire loop as illustrated in FIG. 1.

1. A medical device assembly for cutting body tissue by means of electrotomy, comprising
a generator for a high frequency alternating current which can be delivered by way of a pole and an opposite pole, wherein each pole is connected to at least one respective output terminal of the generator,
a gripping instrument with two gripping arms which each have at least one respective gripping surface and which are connected together in such a way that the gripping surfaces are movable towards each other, wherein the gripping surface is at least partially electrically conductive and each is individually electrically connected to a respective terminal or they are electrically connected to a common terminal for an electrical supply line, and
a cutting instrument which has a handle and a cutting electrode, wherein in operation the cutting electrode is connected to a pole of the generator and both gripping surfaces are connected to the opposite pole of the generator.

2. A medical device assembly as set forth in claim 1 characterized by a converter unit which is connected between the generator and the gripping instrument and the cutting instrument and which is connected on the generator side to the pole and opposite pole of the generator and on the output side to the gripping instrument on the one hand and the cutting instrument on the other hand, in such a way that at a given moment in time both gripping surfaces of the gripping instrument involve a common first potential and the cutting electrode involves a second potential.

3. A gripping instrument for a medical device assembly as set forth in claim 1 comprising two gripping arms which each have at least one respective gripping surface and are connected together in such a way that the gripping surfaces are movable towards each other and the gripping instrument can selectively assume an open or a closed condition, wherein each gripping surface is at least partially electrically conductive, is electrically insulated in relation to the respective other gripping surface and is respectively individually electrically connected to a terminal for a respective electrical supply line, characterized in that the two electrical supply lines are fixedly connected by way of a branching to an electrical supply line to a cutting instrument and the two supply lines are connected by way of the branching to a common supply line for connection to a generator or a converter unit.

4. A gripping instrument for a medical device assembly as set forth in claim 1 comprising two gripping arms which each have at least one respective gripping surface and are connected together in such a way that the gripping surfaces are movable towards each other and the gripping instrument can selectively assume an open or a closed condition, wherein the gripping surface is at least partially electrically conductive, and is electrically connected to a common terminal for an electrical supply line, characterized in that the electrical supply line is fixedly connected by way of a branching to an electrical supply line to a cutting instrument and the two supply lines are connected by way of the branching to a common supply line for connection to a generator or a converter unit.

5. A gripping instrument as set forth in claim 3 characterized in that the gripping surface has a surface structure with raised portions, which permits secure gripping of body pieces.

6. A gripping instrument as set forth in claim 4, characterized in that the gripping surface has a surface structure with raised portions, which permits secure gripping of body pieces.

7. A gripping instrument as set forth in claim 5 characterized in that the raised portions are in the form of biting teeth.

8. A gripping instrument as set forth in claim 6 characterized in that the raised portions are in the form of biting teeth.

9. A gripping instrument as set forth in claim 3 characterized in that a respective gripping surface is a component part of a respective gripping attachment which is to be replaceably secured to the gripping instrument.

10. A gripping instrument as set forth in claim 4 characterized in that a respective gripping surface is a component part of a respective gripping attachment which is to be replaceably secured to the gripping instrument.

11. A gripping instrument as set forth in claim 9 characterized in that a respective gripping attachment is to be clamped onto a respective distal end of a gripping arm.

12. A gripping instrument as set forth in claim 10 characterized in that a respective gripping attachment is to be clamped onto a respective distal end of a gripping arm.

13. A gripping instrument as set forth in claim 9 characterized in that the gripping attachment is made from high-quality steel.

14. A gripping instrument as set forth in claim 10 characterized in that the gripping attachment is made from high-quality steel.

15. A gripping instrument as set forth in claim 3 characterized in that the gripping instrument is designed in the manner of a pair of tweezers.

16. A gripping instrument as set forth in claim 4 characterized in that the gripping instrument is designed in the manner of a pair of tweezers.

17. A gripping instrument as set forth in claim 3 characterized in that the gripping instrument is designed in the manner of biting tongs.

18. A gripping instrument as set forth in claim 4 characterized in that the gripping instrument is designed in the manner of biting tongs.

19. A gripping instrument as set forth in claim 3 characterized in that gripping instrument automatically assumes the open condition without an external force acting thereon.

20. A gripping instrument as set forth in claim 4 characterized in that gripping instrument automatically assumes the open condition without an external force acting thereon.

21. A gripping instrument as set forth in claim 3 characterized in that after setting of the closed condition the gripping instrument retains same or is to be locked in the closed condition.

22. A gripping instrument as set forth in claim 4 characterized in that after setting of the closed condition the gripping instrument retains same or is to be locked in the closed condition.

23. A gripping instrument as set forth in claim 3 characterized in that in the closed condition the gripping instrument is resiliently biased and is to be opened against a spring force.

24. A gripping instrument as set forth in claim 4 characterized in that in the closed condition the gripping instrument is resiliently biased and is to be opened against a spring force.

25. A cutting instrument for a medical device assembly as set forth in claim 1 or claim 2 in which the handle has a gripping surface which is insulated with respect to the cutting electrode and the handle has a terminal for an electrical supply line electrically connected to the cutting electrode (22), wherein the cutting electrode is arranged at a distal end at an electrode shaft and is connected to the handle by way of the electrode shaft, characterized in that the electrical supply line is fixedly connected by way of a branching to an electrical supply line to the gripping instrument and the two supply lines are connected by way of the branching to a common supply line for connection to a generator or a converter unit.

26. A cutting instrument as set forth in claim 25 characterized in that the cutting electrode is pointed.

27. A cutting instrument as set forth in claim 25 characterized in that the cutting electrode is in the form of a wire loop.

28. A cutting instrument as set forth in claim 15 characterized in that cutting electrode or the cutting electrode together with the electrode shaft is replaceably connected to the rest of the cutting instrument, in particular the handle.

29. A cutting instrument as set forth in claim 15 characterized by a press switch which is arranged on the handle and which is to be actuated with a finger and with which an electrical connection to the cutting electrode is to be selectively made or interrupted.

30. A generator for a medical device assembly as set forth in claim 1 characterized by a protective device which responds to the impedance between the pole and the opposite pole falling below or exceeding a limit value or responds to both.

31. A converter unit for a medical device assembly as set forth in claim 2 characterized by a transformer having a primary winding to be connected to the poles of a generator and a secondary winding which is connected to at least one first terminal for a cutting instrument on the one hand and to at least one second terminal for a gripping instrument on the other hand.

32. A converter unit as set forth in claim 31 characterized by a respective capacitor between the secondary winding and the first terminal on the one hand and the secondary winding and the second terminal on the other hand.

33. An electrotomy method in which a gripping instrument is connected to a pole of a generator and a cutting instrument is connected to another pole of the generator, including the method steps:
gripping a piece of body tissue which is to be cut into or severed, with a gripping instrument,
guiding the cutting instrument to a position at which an incision is to be effected, in such a way that a spark flash-over occurs between the cutting instrument and the body tissue at the position of the incision and a current flows between the cutting instrument and the gripping instrument substantially through the piece of body tissue which is to be cut into or severed, and
cutting the body tissue by means of the spark flash-over until the desired incision or severing of the piece of body tissue is completed.

34. A method as set forth in claim 33 wherein a pair of tweezers is used as the gripping instrument.

35. A method as set forth in claim 33 wherein a pointed electrode needle is used as the cutting instrument.

36. A method as set forth in claim 33 wherein shears are used as the cutting instrument.

37. A method as set forth in claim 33 wherein the generator is a high frequency generator for high frequency alternating current.

38. A method as set forth in claim 33 for cutting back an uvula in which the piece of body tissue to be severed is a portion of an uvula.

39. A method as set forth in claim 33 wherein the piece of body tissue to be severed is a portion of a tonsil.

40. A method as set forth in claim 33 wherein the piece of body tissue to be cut into is a velum.

41. A method as set forth in claim 33 used for the treatment of a rhonchopathy.

42. A method as set forth in claim 33 for the treatment of warts in which the body tissue to be severed is a wart and a wire loop is used as the cutting instrument.
